(19) United States
(12) Patent Application Publication
(10) Pub. No.: US 2009/0326360 A1
(43) Pub. Date: Dec. 31, 2009

(54) METHOD FOR ESTIMATING THE GROWTH POTENTIAL OF CEREBRAL INFARCTS

(75) Inventors: Sylvain Baillet, Shorewood, WI (US); Yves Samson, Paris (FR); Nidiyare Hevia-Montiel, Morelos (MX); Charlotte Rosso, Paris (FR); Sandrine Deltour, Vincennes (FR); Éric Bardinet, Gif-Sur-Yvette (FR); Didier Dormont, Vincennes (FR)

Correspondence Address: HARNESS, DICKEY & PIERCE, P.L.C., P.O. BOX 828, BLOOMFIELD HILLS, MI, 48303, US

(73) Assignee: Centre National De La Recherche Scientifique (CNRS), Paris (FR)

(21) Appl. No.: 12/306,867

(22) PCT Filed: Jun. 29, 2007
(86) PCT No.: PCT/FR07/01111

§ 371 Date: Sep. 11, 2009

Related U.S. Application Data
(60) Provisional application No. 60/817,467, filed on Jun. 29, 2006.

(51) Int. Cl. A61B 5/055; G06F 17/10; G06K 9/00

(52) U.S. Cl. 600/410; 703/2; 382/131

(57) ABSTRACT

The invention relates to a method for automatic estimation of the growth potential of cerebral infarcts, particularly in the acute phase, that is to say in the six hours following survival of the stroke. The method includes sequences of diffusion MRI images are obtained, the apparent diffusion coefficient (ADC) is calculated at a multiplicity of points or voxels of the cortical parenchyma, and locating and delimiting the initial infarct and modelling the development of the infarct based on a growth model.

### CROSS-REFERENCE TO RELATED APPLICATIONS

This application is a National Phase entry of International Application No. PCT/FR2007/001111, filed Jun. 29, 2007, claiming priority to U.S. Provisional Patent No. 60/817,467, filed Jun. 29, 2006, both of which are incorporated herein by reference.

### BACKGROUND AND SUMMARY

The invention concerns a method for the automatic estimation of the growth potential of cerebral infarcts, in particular in the acute phase, that is to say within six hours following the occurrence of the stroke. In this regard, the invention concerns the field of cerebral imaging and more particularly the analysis and processing of images obtained by magnetic resonance (MRI) in order to determine the growth potential of cerebral infarcts during their acute phase. The advantage of this procedure is to determine early the risk/benefit ratio presented by the implementation of treatments, effective but aggressive, for combating the propagation of these cerebrovascular attacks.

A few methods used in the form of software tools, not yet standardised, are known from the prior art. Current methods of predicting the growth of cerebral infarcts are from imaging data mainly based on the comparison of the abnormalities in perfusion and diffusion MRI sequences, or on the study of images issuing from the perfusion scanner. In this regard, international patent application no. WO 07/058,632 describes the different steps for obtaining an estimation of the growth potential of the infarct, according to such methods. Despite their major theoretical interest, all these methods have come up against various physiopathological and methodological limitations. Standardising them has proved to be extremely complex and none of them has imposed itself as an indisputable standard. Because of this, implementation thereof remains confined to the context of physiopathological research or possible to large-scale therapeutic tests, in which only a few highly specialised centres can participate.

The only current approaches reported by the literature consist of evaluating a disparity between two types of MRI images acquired sequentially from the patient: a diffusion image that makes it possible to evaluate the extent of the infarct already established and a perfusion image that gives an index of the ischaemic penumbra, that is to say the extent of the neuronal tissue suffering but still able to be saved by therapeutic intervention. The greater the disparity of volume between the regions identified in each series of images, the greater is considered to be the risk of growth. This method, a priori simple and pertinent, is in fact fairly complex to implement. The necessary injection of a contrast substance (for example gadolinium) extends the examination during the perfusion sequence, in particular when venous access is difficult, which is not suited to an emergency clinical context. Moreover, the methods of quantifying perfusion measurements also still remain debated and little standardised and their reproducibility properties are not satisfactory. Finally and especially, no method has made it possible to obtain a satisfactory sensitivity/specificity ratio with an image processing time compatible with emergency use. Analysis of the literature suggests that, though the sensitivity of this method is correct (70% or 80%), its performance in terms of specificity in detection of growth are mediocre.

The aim of the invention is to propose a method that can be implemented in a standardised fashion and makes it possible to automatically, rapidly and reliably predict the growth potential of a cerebral infarct in a patient who has just suffered a stroke. This method makes it possible to supply a tool assisting a therapeutic decision of extreme urgency on an individual scale, or rapid evaluation of new treatments for the pharmaceutical industry on a small group of patients. In the latter case, the effect of a molecule in the test phase can be evaluated rapidly by comparison between the actual growth of the infarct and that predicted by the estimation method according to the invention. If the molecule is effective in reducing the growth of the infarct, the estimation method according to the invention will systematically provide an overestimation of the latter.

The invention uses the fact that, during the acute phase of the infarct, the value of the apparent diffusion coefficient (ADC) is reduced significantly in the regions already infarcted, but also, in a more moderate fashion, in the ischaemic penumbra zone, that is to say the zone liable to be definitively infarcted in the following hours. By automatically detecting the region surrounding the core of the infarct, the mean ADC of which is lower than normal, it is possible to access an estimation of the final size of the infarct. Evaluation of the vital or functional risks run by the patient is then more complete.

Though the regional value of the ADC is central in this method, other criteria are also taken into account in order best to model the growth of the lesion. These supplementary criteria relate to:
the choice of the elements (voxels) of the MRI image that are candidates for inclusion in the growing infarct model;
the regularity of the global shape of the lesion;
the direction of the growth in the volume of the cerebral parenchyma according to the anisotropy properties of the ADC measurements;
match with a numerical probabilistic atlas of the extent of the injured regions for the type of initial occlusion concerned.

More precisely, an object of the invention is a method of estimating the growth potential of cerebral infarcts, the method comprising the following steps: acquisition of diffusion MRI image sequences, calculation of the apparent diffusion coefficient (ADC), at a multitude of points or voxels of the cortical parenchyma, location and delimitation of the initial infarct and modelling of the development of the infarct from a growth model established by the iterative minimisation of a global energy index E defined by a linear combination of elementary energy parameters E_{I} dependent on the intensity of the ADC. This method of estimating the growth potential of cerebral infarcts according to the invention requires only a weighted MRI image sequence in diffusion on a standard MRI imager and normally available in neuroradiology departments. The method does not therefore require the admission intravenously of a contrast agent and the MRI image acquisition time is reduced to less than 5 minutes.

In addition, this method is very simple to implement since the analysis of the 3D maps of the measurement of the ADC in the volume of the cerebral parenchyma absolutely does not depend on the imager used. Finally, the mean regional value of the ADC within the region affected by the final infarct has already been explored in the scientific literature.

Advantageously, the linear combination comprises the following elementary energy parameters E_{I}:
E_{R}, according to which the mean regional value of the ADC within the growing infarct region tends towards a pre-established target value;
E_{S}, according to which the envelope of the infarct has a regular surface;
E_{V}, according to which only the voxels modelling the cerebral tissue are taken into account in calculating this global energy index;
E_{P}, defining the growth probabilities for the infarct and each voxel from empirical schemas;
E_{AN}, defining an anisotropic growth model produced by calculating the local gradient of the apparent diffusion coefficient.

Preferably, these elementary energy parameters E_{R}, E_{S}, E_{V}, E_{P} and E_{AN} are represented by the following mathematical functions:

$${E}_{R} = {( \frac{{i}_{INF} - {\overline{i}}_{INF}}{{σ}_{INF}} )}^{2} ,$$

where
i_{INF} (or respectively σ_{INF}) is the empirical mean (or respectively the standard deviation) of the intensity of the ADC in the growth region INF;
i̅_̅{̅I̅N̅F̅}̅ is a target value of the core regional mean of the ischaemic penumbra zone.

$${E}_{S} = {\sum }_{v ∈ INF} {( \frac{{N}_{v} - N / 2}{δ} )}^{β} ,$$

where
N_{INF} (or respectively N_{IG}) is the number of voxels in the regions INF and IG, respectively;
E_{S} is the regularisation potential according to Ising, well known to the digital image processing community;
N is the total number of voxels adjoining a candidate voxel for inclusion in the region INF (26 for example, in three dimensions) of which N_{V} already belong to INF;
δ and β are fixed scalar parameters.

$${E}_{V} = {\sum }_{v ∈ IG} {( \frac{i ( v ) - {\overline{i}}_{IG}}{{σ}_{IG}} )}^{2} ,$$

where
i(v) is the value of the ADC at voxel v;
i̅_̅{̅I̅G̅}̅ (or respectively σ_{IG}) is an a priori concerning the mean ADC values (or respectively in standard deviation) at the voxels in the growth region IG;
N_{INF} (or respectively N_{IG}) the number of voxels in the regions INF and IG respectively.

$${E}_{p} = {\sum }_{v ∈ INF} p ( v ) ,$$

where
p(v) is the a priori probability that the voxel v belongs to the infarct in its final form.

$${E}_{AN} = \frac{1}{‖ {\vec{V}}_{CDA} ‖ ‖ {\vec{V}}_{INF} ‖} {\sum }_{v ∈ INF} {\vec{V}}_{CDA} ( v ) \cdot {\vec{V}}_{INF} ( v ) ,$$

where
V̅_{CDA} is the stream of gradient vectors defined at each voxel of the ADC map; and
V̅_{INF} the gradients calculated at any point of the binary mask of the growing lesion.
   The energetic minimisation of the state of the lesion as a virtual object makes it possible to introduce several independent parameters and to refine the sensitivity of the results obtained.

According to particular characteristics, the pre-established target value towards which the mean regional value of the ADC tends within the growth region is substantially equal to 740 mm².s⁻¹ or 0.93 times the mean value of the ADC in a controlateral healthy region. This mean regional value of the ADC within the final infarct has already been explored in the scientific literature and is substantially identical from one patient to another. Consequently no adjustment is necessary to obtain good results.

The parameter E_{S} makes it possible to avoid any topological aberration incompatible with the neurophysiopathology of the growing ischaemic lesion. The parameter E_{V} makes it possible to avoid aberrations concerning the value of the ADC at each voxel. If this is too small, it is possibly a case of a voxel at the ADC value that is noisy or affected by artefacts; if it is too high, it is possibly a voxel of the cerebrospinal fluid, undesirable in the lesion.

The parameter E_{P} guides the growth of the infarct through an empirical probability map for each voxel of the image being affected by the lesion. This may be available for each type of original occlusion that led to the ischaemic infarct. Finally, the parameter E_{AN} controls the preferential direction of growth of the lesion in 3D, according to the anisotropy of the distribution of the ADC at each point on the surface of the growing lesion. The infarct will preferentially increase in the direction of local ADC least intensity gradient. The estimation of the final infarct is therefore more reliable.

The invention also concerns a device for implementing a method of estimating the growth potential according to the invention. Thus, once initialised by a mask of the lesion in acute phase, this device makes it possible to obtain a reliable opinion on the growth of the lesion simply, rapidly and automatically.

### BRIEF DESCRIPTION OF DRAWINGS

Other characteristics and advantages of the invention will emerge from the following reading of detailed example embodiments, with reference to the figures, which depict respectively:

FIG. 1, a symbolic diagram of the device for implementing a growth potential estimation method according to the invention;

FIG. 2, a sequential flow diagram for implementing a method of estimating the growth potential of cerebral infarcts according to the invention; and

FIG. 3, a sequence of digital images representing the actual and predicted growths of the cerebral infarct in a patient.

### DETAILED DESCRIPTION

The term “image” employed in the following description refers to the data describing the nature of the cerebral tissue of a patient at many points in space. These “images” therefore consist of a multitude of points for representing a space in two or three dimensions. The digitised “points” forming the image designate voxels (volumetric pixels) or pixels depending on whether or not the image has come from a series of sections exploring part of the three-dimensional space.

The term “map” refers to images, in two or three dimensions, representing the spatial distribution of certain properties of the tissues, also referred to as the “parenchyma”, constituting the head. These maps can come either from databases in order to serve as models that can be adapted to the specificities of each patient, or from the exploitation of the individual data collected during the acquisition of images on a patient. These maps give information on the structure of the brain or on the state of the cerebral tissue of a patient. Advantageously, the maps can be superimposed in order to obtain, on the same image, several complementary information layers.

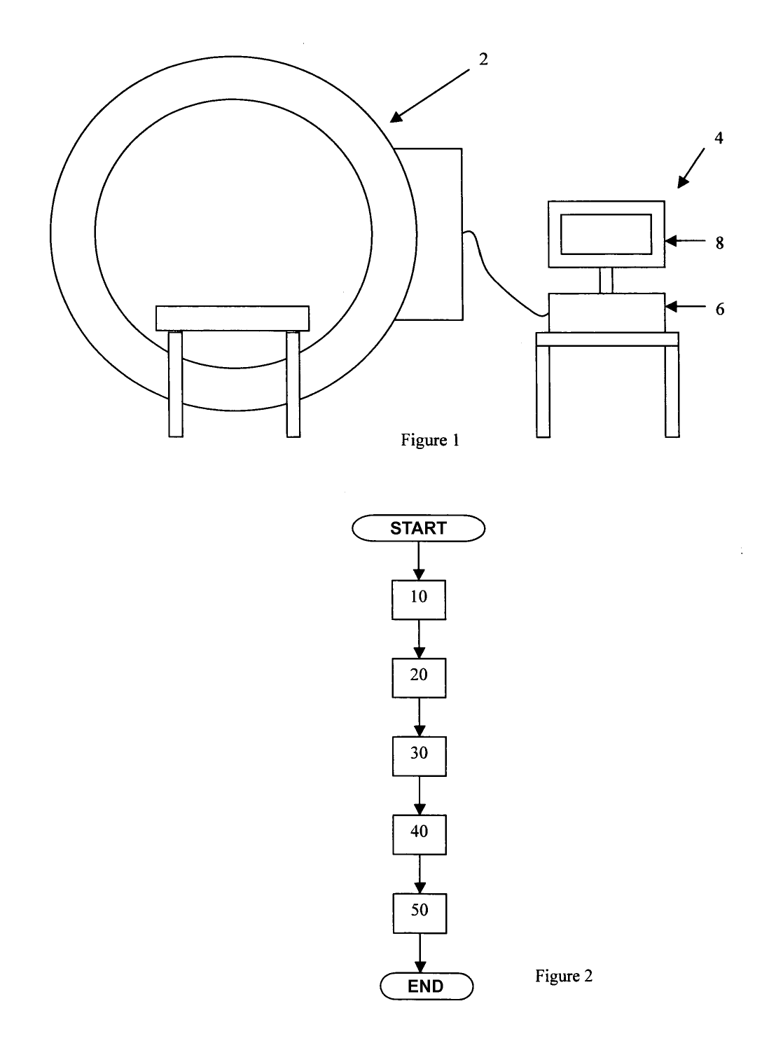

FIG. 1 presents an example embodiment of a growth potential estimation device according to the invention. The device consists in particular of a magnetic resonance imaging apparatus 2 operating at 1.5 teslas or more, able to apply magnetic field gradients in at least six directions in space. The scanner 2 is connected to a workstation 4 provided with a central analysis and image processing unit 6. Thus the scanner 2 transmits the digitised images to the workstation 4, so that the central image analysis and processing unit 6 implements the successive steps (10, 20, 30, 40,) of the method of estimating the growth potential of the infarct according to the invention.

Preferably, this workstation 4 also has a display screen 8 enabling the medical personnel to observe the digital images obtained by means of the scanner 2, to interact with the central image analysis and processing unit 6 and possibly to display the region corresponding to the initial infarct, the growing infarct and the estimated final infarct. According to a particular embodiment, the display screen 8 is a touch screen, which facilitates interaction between the medical personnel and the display screen 8.

FIG. 2 describes the flow diagram of the steps (10, 20, 30, 40, 50) to be followed to implement a method of estimating the growth potential of the infarct according to the invention. The first step 10 of this growth potential estimation method consists of acquiring a sequence of weighted diffusion magnetic resonance digital images (hereinafter referred to as diffusion MRI) according to a standard clinical protocol well known to persons skilled in the art. This protocol uses two sequences of standard digital images applied according to also standardised gradient factors b: b=0 mm².s⁻¹ and b=1000 mm².s⁻¹ in six directions in space. Advantageously, it is possible to use additional gradient values as well as a larger number of acquisition directions, in order to improve the resolution of the information contained at each point in space. Nevertheless, this increase in the resolution takes place to the detriment of the acquisition time, which is extended thereby.

Ideally, the estimation of the risks of propagation of the infarct in the penumbra zones must be made within six hours following the stroke. This is because this estimation enables clinicians to assess pertinently the risk/benefit ratio relating to the treatments, effective but aggressive, that must be carried out as quickly as possible in order to be efficient. The sole use of digital images obtained by diffusion MRI has the advantage of not requiring the intravenous injection of contrast substances or the adjustment of images coming from different additional MRI sequences in order to correct the artefacts due to the movements of the patient during acquisition. This acquisition of digital images by diffusion MRI is therefore simple and rapid to implement, which assist emergency intervention, essential to allow effective treatment of patients.

Advantageously, prior to the analysis of the digital images thus obtained, a step 20 of the growth potential estimation method according to the invention consists of adjusting and normalising the anatomy of the subject in the Talairach reference frame. Alternatively, this adjustment can be made in the MNI reference frame, defined by the Montreal Neurological Institute, or any other system of standardising the anatomy of the cortical parenchyma. This step 20 makes it possible to locate the position at any point in the brain of an individual, with reference to a standardised template. The use of such a reference frame also facilitates the superimposition of the maps issuing from standardised databases with the maps specific to the patient. This type of spatial standardisation can be carried out by a large number of neuroimaging software packages well known to persons skilled in the art.

The following step 30 consists of locating and delimiting the initial infarct from the digital images resulting from the diffusion MRI. According to one embodiment, this step 30 can be carried out by the medical personnel interactively with the central image analysis and processing unit 6. It is then a question of the operator selecting, on the various digital images, a region of the volume of the cerebral parenchyma where the voxel values correspond to a clear hyper-signal in the b1000 diffusion sequences.

According to particular characteristics, this selection is made by thresholding of the images between two measurement values selected by the operator, which enable him to manually contour the injured region in each section or to click in the heart of the lesion so that the image analysis and processing software selects all the voxels relating to this regional seed. Thus the operator specifically selects the aggregate of voxels actually corresponding to the initial ischaemic zone in each section of the volume of diffusion MRI images.

According to an alternative example embodiment, this step 30 is performed automatically by the central image analysis and processing unit 6. The location and delimitation of the initial infarct are then performed according to an automatic process of selecting related regions whose b1000 voxel values significantly exceed a predetermined threshold value. The threshold value depends on the calibration of the MRI scanner 2 in service. Consequently it must be determined empirically. According to a particular embodiment, the threshold value can come from a learning base containing results, collected manually as described above, from around thirty patients.

Another step 40 consists of calculating the value of the apparent diffusion coefficient (ADC) at each point in the sequence of images of the encephalon. The ADC is expressed in mm².s⁻¹. This coefficient is a physical measurement, independent of the site, the type of imager, the magnetic field of the imager and the sequences chosen. Its calculation is entirely standard and well known to persons skilled in the art. It is carried out from the aforementioned b0 and b1000 gradient digital images according to the following formula for each voxel:

$$CDA = \frac{- ln ( Sb 1000 / Sb 0 )}{b 1000 - b 0} , where$$

Sbi is the value of the signal at each voxel considered in the bi sequence.

It should be noted that the steps 30 of locating and delimiting the initial infarct and 40 of calculating the value of the ADC are independent of each other. The order of these steps, with respect to each other, can therefore change without upsetting the results obtained by the growth potential estimation method according to the invention. Finally, the method according to the invention comprises a last step 50, during which the central image analysis and processing unit 6 models the final growth of the infarct from the data collected in the preceding steps (10, 20, 30, 40). The regions affected by the infarct in its initial phase are transferred into the image of the ADC. This transfer takes place automatically and does not require any adjustment between the b1000 and ADC images since they were acquired at the same time. All the voxels belonging to the initial infarct are then used for the initialisation of an automatic process of modelling the growth of the infarct.

The modelling of the growth of the infarct consists of recursively adding voxels to the initially infarcted region, detected during the previous step 30 of the estimation method according to the invention. The underlying model of this modelling therefore consists of virtually enlarging the lesion in its initial state by accumulating voxels from the diffusion MRI images, under certain conditions:
the intensity of the voxels of the diffusion MRI images does not exceed two pre-established threshold values, minimum and maximum;
the mean value of the ADC in the growth region remains less than a pre-established target value, for example during retrospective studies on groups of patients;
the surface of the growing lesion has good regularity properties;
the direction of the growth follows the direction of the minimum gradient of the ADC in the image when the local ADC distribution has a high level of anisotropy; and
the selection of the voxels by matching vis-à-vis a probabilistic digital atlas of the injured regions following an occlusion of a type equivalent to that of the patient being studied.
   Modelling of the growth of the infarct is a digital image analysis and processing problem that can be formalised in many ways (energetic equilibrium model of a system, dynamic process modelled in the form of a set of partial derivative equations, etc). In all cases, and although its algorithmic and software transcription differs, the growth model remains the same.

According to a particular example embodiment of the invention, the growth model is established in an energetic equilibrium formalism. The lesion in its final state is then modelled according to several elementary energy parameters Ei, the linear combination of which defines a global energy index E. By construction, the global energy index E is minimum when the lesion has reached its final growth state.

The elementary energy parameters define respectively:
a control of the deviation of the mean of the ADC within the growth area (E_{R}). This is because the physiopathological models predict that the original mean of the ADC decreases slightly but significantly within the area at risk of future infarction. According to a particular embodiment, the pre-established target value towards which the mean regional value of the ADC tends within the growth region is substantially equal to 740 mm².s⁻¹. Alternatively, this target value is expressed in relation to a control region taken in the parenchyma controlateral to the lesion and is for example equal to 0.93 times the mean value of the ADC in this control region;
a control of the value of the ADC at each voxel candidate for the addition to the growth volume of the infarct so that it remains within acceptable values, predefined on a learning base (E_{V}). Thus only the voxels modelling the cerebral tissue and not the cerebrospinal fluid are taken into account in the calculation of this functional energy equation;
a check on the regularity of the form of the external surface of the growing infarct E_{S};
a check on the direction of the growth so that it takes place according to the minimum ADC spatial variation gradient E_{AN};
a check on the match of the growth with a probabilistic atlas of the affliction of cerebral regions by an ischaemic infarct E_{P}.

These elementary energy parameters E₁ are combined according to the following formula, defining the global energy index E:

$$E = {\sum }_{v ∈ INF} ( 1 - FA ( v ) ) \cdot {E}_{IS} ( v ) - γ \cdot FA ( v ) \cdot {E}_{AN} ( v ) + θ \cdot Ep ( v ) ,$$

where
FA is the mean fractional anisotropic coefficient, the definition of which is known to persons skilled in the art, characterising the distribution of the ADC around the voxels at the surface of the lesion currently growing;
θ and γ are fixed scalar parameters;
E_{AN} is the total correlation index between the stream of gradient vectors
   defined at each voxel of the ADC map V̅_{CDA}, well known to persons skilled in art, and the gradients calculated at any point of the binary mask of the growing lesion V̅_{INF}:

$${E}_{AN} = \frac{1}{‖ {\vec{V}}_{CDA} ‖ ‖ {\vec{V}}_{INF} ‖} {\sum }_{v ∈ INF} {\vec{V}}_{CDA} ( v ) \cdot {\vec{V}}_{INF} ( v )$$

E_{P} is a matching factor established between a probalistic atlas and the current growth of the lesion. The probability P_{(V)} that the voxel v belongs to the infarct in its final form is obtained empirically, on patients that have suffered a similar type of infarct, originating in the same initial occlusion. The matching factor E_{P} totals the probabilities of affliction of each voxel belonging to the growing lesion, as estimated at each iteration:

$${E}_{p} = {\sum }_{v ∈ INF} p ( v ) ;$$

$$where$$

E_{IS} is a linear combination of three elementary energy parameters:

$${E}_{IS} = {E}_{R} + {δ}_{V} {E}_{V} + {β}_{S} {E}_{S} , \\ with$$

$${E}_{R} = {( \frac{{i}_{INF} - {\overline{i}}_{INF}}{{σ}_{INF}} )}^{2} , \\ {E}_{V} = {( \frac{i ( v ) - {\overline{i}}_{IG}}{{σ}_{IG}} )}^{2} , \\ {E}_{S} = {( \frac{{N}_{v} - N / 2}{δ} )}^{β}$$

I_{INF} (or respectively σ_{INF}) is the empirical mean (or respectively the standard deviation) of the intensity of the ADC in the growth region INF;
ī_{INF} is a target value of the core regional mean of the ischaemic penumbra zone;
i(v) is the value of the ADC at the voxel v;
ī_{IG} (or respectively σ_{IG}) is an a priori concerning the mean ADC values (or respectively standard deviation values) at the voxels in the growth region IG;
N_{INF} (or respectively N_{IG}) the number of voxels in the regions INF and IG, respectively;
E_{S} is the regularisation potential according to Ising, well known to the scientific community specialising in the processing of digital images;
N is the total number of voxels adjoining a voxel candidate for inclusion in the INF region (26 for example in three dimensions), of which N_{V} already belong to INF;
δ and β are fixed scalar parameters.

The modelling of the growth is carried out iteratively by successive accumulation of the voxels immediately adjoining the growing region. Accumulation ends when the global energy index E of the virtual lesion is minimised. The voxels selected consequently constitute the estimated final infarct region.

According to an alternative example embodiment, it is also possible to consider a growth model that uses variational dynamic formalism of the “level line” or level set type, well known to persons skilled in the art, in particular by the resolution of differential equations relating to identical voxel selection criteria. The use of such a formalism makes it possible to end up exactly with the same results in terms of quality of modelling and anticipation of the final growth of the lesion. Only performance differences in terms of calculation time could distinguish these two alternative embodiments.

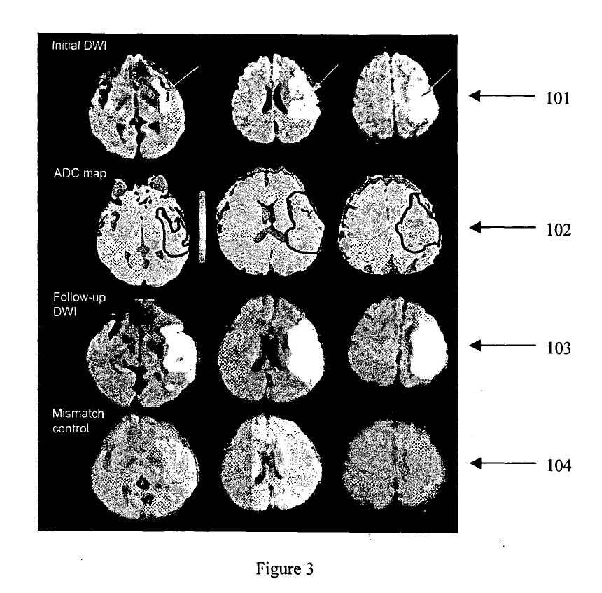

Finally, FIG. 3 presents several sequences of images 101, 102, 103, 104 depicting the actual and predicted growths of the cerebral infarct in a patient. According to one embodiment, these images are displayed by the medical personnel on the display screen 8. The first sequence of images 101 depicts three sections illustrating the cortical parenchyma, obtained during the initial step 10 of acquiring diffusion MRI images. The infarct already formed appears on this first sequence of images in clear white hypersignal.

The second sequence of images 102 depicts the map of the apparent diffusion coefficient encoded in false colours. The growth prediction obtained by the estimation method according to the invention is incorporated on this map and predicts a final infarct size contoured in black. The third sequence of images 103 depicts three illustrative sections obtained by diffusion MRI 24 hours after the stroke. The growth phase of the infarct has then ended and the actual final size of this infarct is easily identifiable in the same way as in the first sequence of images, in white hypersignal. Finally, the fourth sequence of images 104 makes it possible to obtain, by a visual check, a match between the size of the final infarct, in white hypersignal as in the third sequence of images, and the automatic prediction obtained by the estimation method according to the invention, shown in blue.

Naturally the invention is not limited to the example embodiments described and depicted above. It is understood that a person skilled in the art is in a position to implement variants of the invention without for all that departing from the scope of this patent.

1. A method of estimating the growth potential of cerebral infarcts, the method comprising:
acquiring sequences of diffusion MRI images;
calculating the apparent diffusion coefficient (ADC) at a multitude of points or voxels on the cortical parenchyma;
locating and delimiting the initial infarct; and
modelling the change in the infarct from a growth model established by the iterative minimisation of a global energy index E defined by a linear combination of elementary energy parameters dependent on the intensity of the ADC.

2. The method of estimation the growth potential of cerebral infarcts according to claim 1, in which the linear combination comprises an elementary energy parameter E_{R} according to which the mean regional value of the ADC within the growth region tends towards a pre-established target value.

3. The method of estimating the growth potential of cerebral infarcts according to claim 2, in which the elementary energy parameter E_{R} is represented by the following mathematical function:
$${E}_{R} = {( \frac{{i}_{INF} - {\overline{i}}_{INF}}{{σ}_{INF}} )}^{2} , \\ where$$
i_{INF} (or respectively σ_{INF}) is the empirical mean (or respectively the standard deviation) of the intensity of the ADC in the growth region INF; and
ī_{INF} is a target value of the core regional mean of the ischaemic penumbra zone.

4. The method of estimating the growth potential of cerebral infarcts according to claim 3, in which the pre-established target value towards which the mean regional value of the ADC tends within the growth region is substantially equal to 740 mm².s⁻¹.

5. The method of estimating the growth potential of cerebral infarcts according to claim 1, in which the pre-established target value towards which the mean regional value of the ADC tends within the growth region is substantially equal to 0.93 times the regional mean value of the ADC in a controlateral healthy region.

6. The method of estimating the growth potential of cerebral infarcts according to claim 1, in which the linear combination comprises an elementary energy parameter E_{S} according to which the envelope of the infarct has a regular surface.

7. The method of estimating the growth potential of cerebral infarcts according to claim 6, in which the elementary energy parameters E_{S} is represented by the following mathematical function:
$${E}_{S} = {\sum }_{v ∈ INF} {( \frac{{N}_{v} - N / 2}{δ} )}^{β}$$
$$where$$
N_{INF} (or respectively N_{IG}) is the number of voxels in the regions INF and IG, respectively;
E_{S} is the regularisation potential according to Ising, well known to the digital image processing community;
N is the total number of voxels adjoining a candidate voxel for inclusion in the region INF (26 for example, in three dimensions) of which N_{V} already belong to INF; and
δ and β are fixed scalar parameters.

8. The method of estimating the growth potential of cerebral infarcts according to claim 1, in which the linear combination comprises an elementary energy parameter E_{V} according to which only the voxels modelling the cerebral tissue are taken into account in calculating this global energy index E.

9. The method of estimating the growth potential of cerebral infarcts according to claim 8, wherein elementary energy parameter E_{V} is represented by the following mathematical function:
$${E}_{V} = {\sum }_{v ∈ IG} {( \frac{i ( v ) - {\overline{i}}_{IG}}{{σ}_{IG}} )}^{2} , \\ where$$
i(v) is the value of the ADC at voxel v;
ī_{IG} (or respectively σ_{IG}) is an a priori concerning the mean ADC values (or respectively a standard deviation) at the voxels in the growth region IG; and
N_{INF} (and respectively N_{IG}) the number of voxels in the regions INF and IG respectively.

10. The method of estimating the growth potential of cerebral infarcts according to claim 1, in which the linear combination comprises an elementary energy parameter E_{P} defining the probabilities of growth of the infarct at each voxel from empirical schemes.

11. The method of estimating the growth potential of cerebral infarcts according to claim 10, wherein elementary energy parameter E_{P} is represented by the following mathematical function:
$${E}_{p} = {\sum }_{v ∈ INF} p ( v ) , \\ where$$
p(v) is the a priori probability that the voxel v belongs to the infarct in its final form.

12. The method of estimating the growth potential of cerebral infarcts according to claim 1, in which the linear combination comprises an elementary energy parameter E_{AN} defining an anisotropic growth model, achieved by calculating the local gradient of the apparent diffusion coefficient.

13. The method of estimating the growth potential of cerebral infarcts according to claim 12, wherein elementary energy parameter E_{AN} is represented by the following mathematical function:
$${E}_{AN} = \frac{1}{‖ {\vec{V}}_{CDA} ‖ ‖ {\vec{V}}_{INF} ‖} {\sum }_{v ∈ INF} {\vec{V}}_{CDA} ( v ) \cdot {\vec{V}}_{INF} ( v ) , \\ where$$
V̅_{CDA} is the stream of gradient vectors defined at each voxel of the ADC map; and
V̅_{INF} the gradients calculated at any point of the binary mask of the growing lesion.

14. The method of estimating the growth potential of cerebral infarcts according to claim 1, in which the step of locating and delimiting the initial infarct is automatic.

15. A device for estimating the growth potential of cerebral infarcts implementing a method according to claim 1.
